(19) United States
(12) Patent Application Publication
(10) Pub. No.: US 2016/0324479 A1
(43) Pub. Date: Nov. 10, 2016

(54) Handheld Gas Sensing Device and Sensing Method Thereof

(71) Applicant: NATIONAL TSING HUA UNIVERSITY, Hsinchu (TW)

(72) Inventors: Kea-Tiong TANG, Taipei City (TW); Shih-Wen CHIU, Zhubei City (TW); Chung-Hung SHIH, Taipei City (TW); Li-Chun WANG, Taoyuan City (TW); Hsin CHEN, Hsinchu City (TW); Yi-Wen LIU, Hsinchu City (TW); Chia-Min YANG, Hsinchu City (TW); Da-Jeng YAO, Taipei City (TW)

(21) Appl. No.: 14/831,664

(22) Filed: Aug. 20, 2015

(30) Foreign Application Priority Data
May 6, 2015 (TW) 104114472

(51) Int. Cl. A61B 5/00; A61B 5/097; A61B 5/08

(52) U.S. Cl. CPC A61B 5/7264; A61B 5/082; A61B 5/097; A61B 5/7246; A61B 5/742; A61B 2562/0271; A61B 2562/029; A61B 2562/0204; A61B 2562/0285

(57) ABSTRACT

A handheld gas sensing device and sensing method thereof are provided. The handheld gas sensing device includes a plurality of gas sensing chips and a gas collector. The plurality of gas sensing chips respectively include a sensing array, a sensing interface circuit, a microcontroller, and a memory. The gas signal is determined by the gas adsorption of the sensing array. The gas signal is converted to a visible operand by using the sensing interface circuit. The visible operand is projected to a hidden operand by utilizing the calculation of Continuous Restricted Boltzmnan Machine (CRBM). The plurality of gas sensing chips are connected with each other to do the multi-layer calculation of CRBM. The probability of the to-be-detected gas is obtained. The result is recorded in the memory.

### CROSS-REFERENCE TO RELATED APPLICATION

This application claims the benefit of Taiwan Patent Application No. 104114472, filed on May 6, 2015, in the Taiwan Intellectual Property Office, the disclosure of which is incorporated herein in its entirety by reference.

### BACKGROUND OF THE INVENTION

1. Field of the Invention

The present disclosure generally relates to a handheld gas sensing device and sensing method thereof, in particular to a handheld gas sensing device and sensing method thereof for detecting gas by cascading a plurality of sensing chips.

2. Description of the Related Art

Currently, the standard gas sensing inspection mode is to analyze the collected gases by the Gas Chromatography Mass Spectrophotometer (GC-MC) or Fourier Transform Infrared Spectrometry (FT-IR) in laboratory. Although the components of the gas can be accurately analyzed through the large instruments in the laboratory, such treatment may lack of instantaneity and popularity. As a result, developing the portable, rapid detecting and economical gas sensing device is indeed imperative.

Generally, the gas analysis includes sensing the external gases or gases exhaled from human body, and such detection result may demonstrate situations of the environmental pollution or the human body health. The current gas sensing device such as electronic nose has a larger volume, but it is still limited to the specific target gas detection and not able to accurately detect various gases and the masses are therefore dissatisfied with the portability and usage of the current gas sensing device. In addition, when collecting the external gases or gases exhaled from human body, the sample of the collected gas may be affected to cause erroneous recognition result due to the temperature and humidity.

As mentioned above, the inventor of the present disclosure has been mulling the technical problem over and then designs a gas sensing device and gas sensing method thereof to aim to shortcomings of the existing technique so as to promote the industrial practicability.

### SUMMARY OF THE INVENTION

In view of the foregoing technical problem, the objective of the present disclosure provides a handheld gas sensing device and sensing method thereof to resolve the problem concerning that the existing gas sensing device is incapable of sensing gas instantly and commonly.

According to one objective of the present disclosure, a handheld gas sensing device is provided, which includes a plurality of gas sensing chips and a gas collector. The plurality of gas sensing chips include a sensing array, a sensing interface circuit, a microcontroller and a memory. The sensing array includes a sensing thin film and a sensor. The sensing thin film is provided for absorbing gas, such that a to-be-detected gas exhaled from mouth or nose is absorbed and a to-be-detected gas signal is produced by the sensor. The sensing interface circuit is connected to the sensing array and converting the to-be-detected gas signal into a visible operand. The microcontroller is connected to the sensing interface circuit and projecting the visible operand to a hidden operand by utilizing a calculation of Continuous Restricted Boltzman Machine (CRBM) to calculate a distributed result of the to-be-detected gas and comparing the distributed result with a probability model of a target gas to obtain a probability for recognizing the to-be-detected gas with respect to the target gas. The memory is recording the target gas and the probability model of the target gas and recording the probability of a comparison result. The gas collector is directly collecting the to-be-detected gas exhaled from mouth or nose, and the to-be-detected gas is transferred to the sensing array through a transfer pipeline. The plurality of gas sensing chips are cascaded to each other such that the microcontrollers of the plurality of gas sensing chips collaboratively work together, and the hidden operand of one of the gas sensing chips produced through projection and calculation is served as the visible operand of another gas sensing chip to be projected again to produce another hidden operand, and the distributed result produced by a multi-layer calculation of Continuous Restricted Boltzman Machine is compared with the probability model to obtain the probability for recognizing the to-be-detected gas with respect to the target gas.

Preferably, the plurality of gas sensing chips may correspond to respective target gases, and the plurality of the gas sensing chips are connected in parallel with each other to simultaneously obtain the probability for recognizing the to-be-detected gas with respect to the respective target gases.

Preferably, the handheld gas sensing device may further include a temperature-humidity sensor including a resistance having a temperature coefficient and a humidity coefficient, and a measured value of the resistance is producing a temperature-humidity signal to correct the visible operand of the to-be-detected gas according to the temperature-humidity signal.

Preferably, the probability model may include a classifier and the classifier classifies the distributed result and compares the distributed result with the probability model to obtain the probability for recognizing the to-be-detected gas with respect to the target gas.

Preferably, the classifier may classify the distributed result by a linear programming model or a support vector model.

Preferably, the sensing thin film may include a plurality of nanoporous carbon materials and a polymer grows in pores of the nanoporous carbon materials to absorb the to-be-detected gas.

Preferably, the sensor may include a conductive polymer gas sensor and a surface acoustic wave sensor.

Preferably, the handheld gas sensing device may further include a display device to display the probability for recognizing the to-be-detected gas with respect to the target gas.

According to another objective of the present disclosure, a gas sensing method is provided, which includes following step: directly collecting a to-be-detected gas exhaled from mouth or nose by a gas collector of a handheld gas sensing device and transporting the to-be-detected gas to a plurality of gas sensing chips through a transfer pipeline, and each of the plurality of gas sensing chip including a sensing array; absorbing the to-be-detected gas by a sensing thin film of the sensing array and producing a to-be-detected gas signal by a sensor; converting the to-be-detected gas signal into a visible operand and transmitting the visible operand to a microcontroller by a sensing interface circuit; projecting the visible operand to a hidden operand by utilizing a calculation of Continuous Restricted Boltzman Machine (CRBM) to calculate a distributed result of the to-be-detected gas; cascading the plurality of gas sensing sensors with each other to enable microcontrollers of the plurality of gas sensing sensors to work together collaboratively and producing the hidden operand of one of the gas sensing chips through projection and calculation to serve as the visible operand of another gas sensing chip so as to be projected again to produce another hidden operand, and producing the distributed result by a multi-layer calculation of Continuous Restricted Boltzman Machine; comparing the distributed result with a probability model stored in a memory to obtain a probability for recognizing the to-be-detected gas with respect to a target gas.

Preferably, the plurality of gas sensing chips may correspond to respective target gases, and the plurality of the gas sensing chips are connected in parallel with each other to simultaneously obtain the probability for recognizing the to-be-detected gas with respect to the respective target gases.

Preferably, the gas sensing method may further include following step: producing a temperature-humidity signal by a temperature-humidity sensor to correct the visible operand of the to-be-detected gas, and the temperature-humidity sensor comprising a resistance having a temperature coefficient and a humidity coefficient.

Preferably, the distributed result of the to-be-detected gas may be classified by a classifier and the distributed result is compared with the probability model.

Preferably, the classifier may classify the distributed result by a linear programming model or a support vector model.

Preferably, the sensing thin film may include a plurality of nanoporous carbon materials and a polymer grows in pores of the nanoporous carbon materials to absorb the to-be-detected gas.

Preferably, the sensor may include a conductive polymer gas sensor and a surface acoustic wave sensor.

Preferably, the probability for recognizing the to-be-detected gas with respect to the target gas may be displayed by a display device.

As mentioned previously, the handheld gas sensing device and sensing method thereof disclosed in the present disclosure may have one or more following advantages.

(1) The handheld gas sensing device and sensing method thereof integrate the sensing device on the chips and cascade the plurality of gas sensing chips so as to be expanded, such that the handheld gas sensing device and sensing method thereof are capable of sensing various target gases. In addition, the chips are connected to work together collaboratively to group and recognize the sensing signals of the sensor more precisely.

(2) The handheld gas sensing device and sensing method thereof are able to calculate the probability for recognizing the to-be-detected gas with respect to the respective target gases, not merely determining the sensed result by one decision. Hence, it is able to avoid that different target gases having signal superposition are neglected to lead to a misjudgment of the sensed result.

(3) The handheld gas sensing device and sensing method thereof can measure the temperature and humidity signals of the to-be-detected gas to compensate the gas sensing signal through the temperature and humidity signals so as to avoid the negative effect of the sensed result when collecting gas.

### BRIEF DESCRIPTION OF THE DRAWINGS

FIG. 1 is a block diagram of the handheld gas sensing chip of the present disclosure.

FIG. 2 is a schematic diagram of the multi-layer calculation of CRBM of the present disclosure.

FIG. 3 is a schematic diagram showing the expert system formed by connecting the plurality of gas sensing chips of the present disclosure.

FIG. 4 is a schematic diagram showing the cascade of the plurality of gas sensing chips of the present disclosure.

FIG. 5 is a flow chart of the gas sensing method of the present disclosure.

FIG. 6 is a schematic diagram of the handheld gas sensing device of the present disclosure.

### DETAILED DESCRIPTION OF THE PREFERRED EMBODIMENTS

In order to facilitate the understanding of the technical features, the contents and the advantages of the present disclosure, and the effectiveness thereof that can be achieved, the present disclosure will be illustrated in detail below through embodiments with reference to the accompanying drawings. On the other hand, the diagrams used herein are merely intended to be schematic and auxiliary to the specification, but are not necessary to be true scale and precise configuration after implementing the present disclosure. Thus, it should not be interpreted in accordance with the scale and the configuration of the accompanying drawings to limit the scope of the present disclosure on the practical implementation.

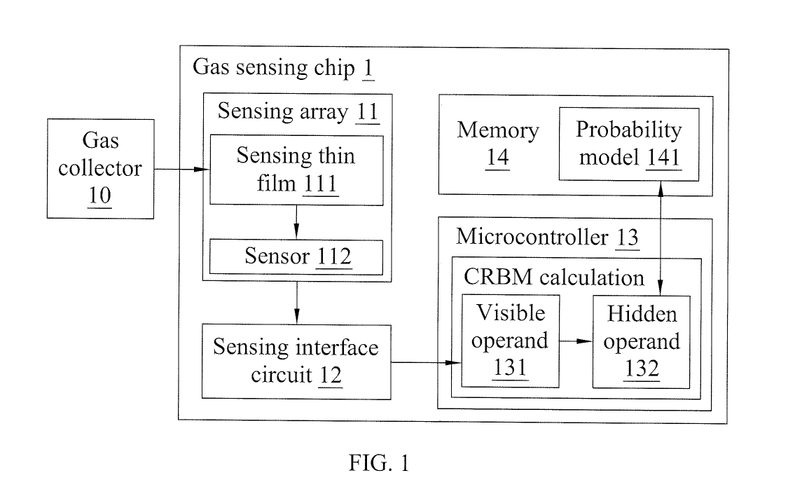

Please refer to FIG. 1 which is a block diagram of the handheld gas sensing chip of the present disclosure. As shown in FIG. 1, a gas sensing chip 1 includes a sensing array 11, a sensing interface circuit 12, a microcontroller 13, and a memory 14. The sensing array 11 is connected to a gas collector 10. A tube of the gas collector 10 is provided to enable user to hold in the mouth and to blow gas to therein, such that the exhaled gas can be collected by the gas collector 10. Alternatively, a mask is provided to cover user's mouth or nose to collect the exhaled gas, and then the collected to-be-detected gas is transported to a sensing thin film 111 of the sensing array 11 through the transfer pipeline. The sensing thin film 111 includes a plurality of nanoporous carbon materials and a polymer with gas absorbability grows in pores of the nanoporous carbon materials to absorb the to-be-detected gas. The nanoporous carbon material is made according to the structure of human's nasal cavity, and the polymer with gas absorbability is applied to absorb the to-be-detected gas. The reaction produced by different polymer and the target gas is used to detect signal of the to-be-detected gas. The sensing thin film 111 may also apply the polymer hollow mesoporous carbon spheres as the structure, size of the carbon spheres is 80 to 120 nm, and thickness of the thin film is 10 nm. The sensing thin film 111 is connected to a sensor 112 to measure a reaction value when the gas molecules are absorbed. The sensor may be a conductive polymer gas sensor which uses the changes of resistance of the sensing material to serve as a detection signal or a surface acoustic wave sensor which is to detect by the oscillation frequency.

In addition, the sensing array 11 further includes a temperature-humidity sensor. The temperature and humidity of the to-be-detected gas may change the resistance to affect the sensed result when the sensing array 11 is reacting, and thus, the temperature-humidity sensor is provided to measure the temperature and humidity. For example, the resistances having a temperature coefficient and a humidity coefficient are respectively arranged, and when the to-be-detected gas is passing, a temperature sensing signal and a humidity sensing signal produced by the change of the resistances and a reciprocal effect value of the influence values of temperature and humidity variations measured in advance are used to compensate the detection signal of the to-be-detected gas. The formula of the sensing signal compensation is stated as follows.

s^{(T}^{ₙ}^{,H}^{ₙ}⁾=s^{(T}^{₀}^{,H}^{₀}⁾+f_{T}(ΔH)+f_{H}(ΔH)+f_{TH}(ΔT,ΔH) (1)

S={s₁, s₂ . . . s_{d}} is a d-dimensional sensing signal, s^{(To, Ho)} denotes the sensing signal at temperature T₀ and humidity Hₒ. When the temperature and humidity variations are respectively Tₙ and Hn, the sensing signal s^{(Tn, Hn)} needs to be added the influential value of the temperature variation f_{T} (ΔT), the influential value of the humidity variation f_{H}(ΔH) and the influential value of the reciprocal effect value of the influence values of temperature and humidity variations f_{TH}(ΔT, ΔH). The functional relations of the aforementioned influential values may be made by the reaction of the temperature and humidity measured by the sensor in advance. For example, the model inferred by the relation between the resistivity and temperature. As a result, when detecting the gas, the temperature-humidity sensor respectively measures the rate of change of the to-be-detected gas and the predetermined temperature and humidity, and then the sensing signal is compensated according to the variations, so as to obtain the signal of the to-be-detected gas more precisely.

Please refer to FIG. 1 again. The signal sensed by the sensor 112 passes the sensing interface circuit 12 to conduct the conversion, and the sensing interface circuit 12 converts the signal sensed by the sensor 112 into a voltage signal and then the voltage signal is converted into a digital code by an analog-to-digital (A/D) converter, and then the digital code is provided to the microcontroller 13 for the follow-up identification and analysis. The converted signal forms a visible operand 131 of the Continuous Restricted Boltzman Machine (CRBM). The visible operand 131 is projected to a hidden operand 132 through the data projection to achieve the effect of grouping and dimension reduction. The CRBM has been achieved as the modularized analog chip system, and the reference can be referred as follows. Lu, C. C. and Chen, H., “A Scalable and Programmable Probabilistic Generative Model in VLSI”, submitted to IEEE Trans. on Neural Networks, 2010, and Lu, C. C. and Chen, H., “Current-mode Computation with Noise in a Scalable and Programmable Probabilistic Neural VLSI System”, and so on. The CRBM-based stochastic neural network chip disclosed in the present disclosure is integrated in the recognition chip of the gas sensing chip, so that the gas recognition chip can process biomedical signals with many noises and large variance. The CRBM used for the pretreatment of the gas signal can amplify the difference of different types of signals stably, and learn the main characteristic of the signal distribution to obtain an output with dimension smaller than that of the original signal. The computation of the back-end processor can be reduced effectively. In addition, the CRBM has a learning ability capable of timely and appropriately adjusting the model parameters to maintain reliable recognition ability for the different sensors, different gas compositions, or the shift of the sensor resulted from a long time of usage. Here, the obtained distributed result of the hidden operand 132 is stored in the memory 14 for being served as a probability model 141 of the target gas, such that the probability model 141 can be corrected through the learning ability. When detecting gas, the probability of the to-be-detected gas corresponding to the probability model 141 is calculated, so as to determine whether the to-be-detected gas belongs to the target gas.

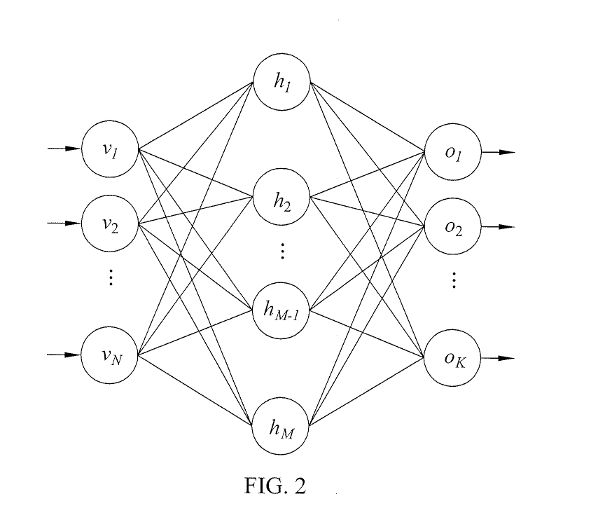

Please refer to FIG. 2 which is a schematic diagram of the multi-layer calculation of CRBM of the present disclosure. As shown in FIG. 2, N^{th} visible operands V₁, V₂ . . . Vₙ are generated when the to-be-detected gas is detected by the sensing array, and these signals are projected to M^{th} hidden operands h₁, h₂ . . . h_{M} after being calculated by CRBM. The conventional CRBM only has one layer calculation of visible operand and one hidden operand. If N is greater than M, the projected data naturally achieves to the distribution status which is able to be grouped, and then the type of the target gas can be determined after being compared with the data of the target gas. However, the complicated sensing signals fails to be performed the follow-up data grouping only by a single layer calculation, such as the gases related to the pneumonia bacteria. Take the Chronic Obstructive Pulmonary Disease (COPD) as an example, it includes Carbon monoxide, Nitric oxide, Ethane, Isoprene, Pentane, 2-Methylpentane, Heptane, Octane, Ethylbenzene, Xylenes, Styrene, 1,2,4-Trimethylbenzene, and Decane which all belong to the target gases related to the COPD. Hence, when doing the sensing calculation, the single layer calculation of CRBM fails to effectively group the types of the aforementioned gases, and the distribution status of the to-be-detected gas has to be projected to hidden operand of a higher layer calculation, such that the data grouping is able to be achieved effectively. Take a three-layer calculation of CRMB for example, the hidden operands h₁, h₂ . . . h_{M} are projected again to obtain K^{th} hidden operands o₁, o₂ . . . o_{K} of the second layer calculation, and after being projected and analyzed through the hidden layer by two times, the various data is able to be grouped so as to be served as an output of the to-be-detected gas. A classifier is provided to recognize the types of the target gases by linear partition. The classifier is used to classify the distributed result by a linear programming model or a support vector model, and the linear partitioned plane maximizes the various data and minimizes the variety of the same data, so as to obtain the optimal classified result. As the integrated circuit applied to do the calculation of CRBM is embedded in the system chip, cascading the plurality of gas sensing chips enables the microcontrollers of the chips to work together collaboratively to do a multi-layer calculation of CRBM, and thus, the data of complicated gas can be recognized effectively. In addition, the aforementioned classifier may also be accomplished by cascading the chips.

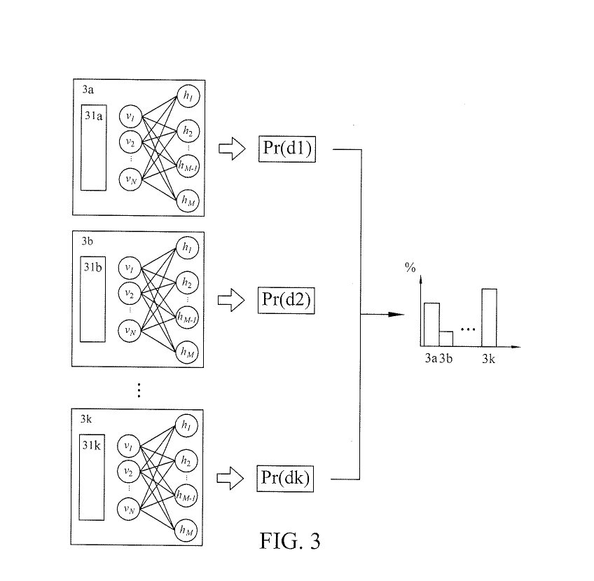

Please refer to FIG. 3, which is a schematic diagram showing the expert system formed by connecting the plurality of gas sensing chips of the present disclosure. As shown in FIG. 3, a handheld gas sensor 3 includes cascading a first gas sensing chip 3a, a second gas sensing chip 3b to K^{th} gas sensing chip 3k. The K^{th} gas sensing chip 3k is applied to detect various target gases, such as the gases related to the COPD as mentioned above. In the sensing arrays 31a, 31b . . . 31k, the sensing thin films made of different polymeric materials are respectively applied to detect the target gases correspondingly. The gas sensing chips obtain the visible operands V₁, V₂ . . . V_{N} from the to-be-detected gas d, respectively, and then the visible operands V₁, V₂ . . . V_{N} are projected to the hidden operands h₁, h₂ . . . h_{M} after being performed the calculation of CRBM. An energy of the to-be-detected gas d corresponding to each chip in the CRBM model is calculated and then the probability Pr(d₁), Pr(d₂) . . . Pr(dₖ) for recognizing the to-be-detected gas with respect to the respective target gases is further calculated. The energy value E(d) and the probability Pr(d) corresponding to the to-be-detected gas d are calculated by using the following formula.

$$\begin{matrix}E ( d ) = - \frac{1}{2} {\sum }_{i \neq j} {w}_{ij} {v}_{i} {h}_{j} + {\sum }_{j} \frac{1}{{a}_{j}} {\int }_{0}^{{s}_{j}} {ϕ}^{- 1} ( s ) d s & ( 2 ) \\ Pr ( d ) = \frac{1}{2} {e}^{- E ( d )} & ( 3 )\end{matrix}$$

Vᵢ is the value of i^{th} visible operand, h₁ is the value of the j^{th} hidden operand, wᵢᵢ is the connection weight therebetween, aⱼ is the sigmoid inclination of the function of the hidden operand hⱼ, φ(x) is the sigmoid function. After calculating the probability Pr(d₁), Pr(d₂) . . . Pr(dₖ) for recognizing the to-be-detected gas with respect to the respective target gases, a reciprocal diagram 32 showing the probability for recognizing the to-be-detected gas with respect to respective k^{th} gas sensing chips is shown on the display device of the gas sensing device, not directly determining that the to-be-detected gas belongs to certain specific target gas, and the user is able to determine the percentage for recognizing the to-be-detected gas with respect to the specific gas through the probability. Such recognition method is to use the chips, which are respectively characterized of sensing certain target gas, to form multiple expert systems to recognize the types of the gases and avoid whether the to-be-detected gas belongs to certain associated gas through a decisive recognition manner, such that the target gas having the same sensing signal may be deleted. As a result, the gas sensing device is free from causing errors.

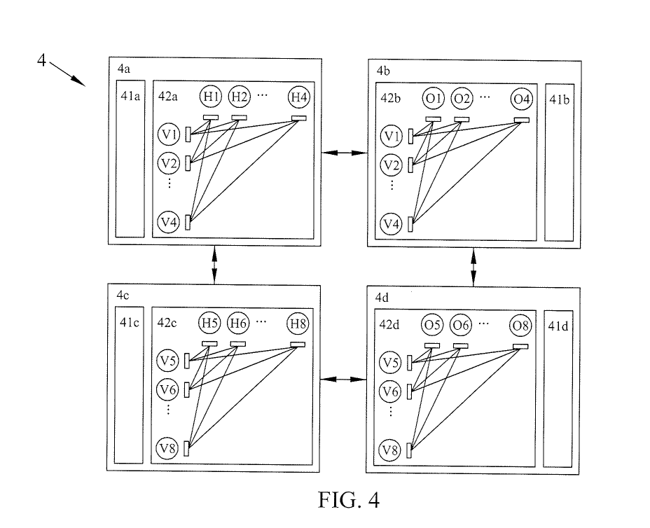

Please refer to FIG. 4, which is a schematic diagram showing the cascade of the plurality of gas sensing chips of the present disclosure. As shown in FIG. 4, the handheld gas sensing sensor 4 includes a first gas sensing chip 4a, a second gas sensing chip 4b, a third gas sensing chip 4c and a fourth gas sensing chip 4d which are cascaded with each other. The four gas sensing chips respectively include the sensing arrays 41a, 41b, 41c and 41d and the microcontrollers 42a, 42b, 42c and 42d. Each of the chips includes four visible operands and four hidden operands. The first gas sensing chip 4a and the third gas sensing chip 4c are connected in parallel with each other, enabling the sensing device having eight visible operands (V1-V8) and eight hidden operands (H1-H8). The first gas sensing chip 4a and the third gas sensing chip 4c respectively detect different target gases to form the connection of two expert systems. The first gas sensing chip 4a is cascaded with the second gas sensing chip 4b and the third gas sensing chip 4c is cascaded with the fourth gas sensing chip 4d to form a three-layer network structure of CRBM. The eight visible operands (V1-V8) are projected to the eight hidden operands (H1-H8), and the eight hidden operands (H1-H8) are served as the visible operand of the second gas sensing chip 4b and the fourth gas sensing chip 4d. The eight visible operands (V1-V8) are projected to the hidden operand of the second-layer calculation to form eight output operands (O1-O8). By calculating the foregoing probability model of the eight output operands (O1-O8) with respect to the target gases, it can obtain the probability for recognizing the to-be-detected gas with respect to the target gas. Such connection increases the types of the recognized gases by different sensing arrays, that is, to increase the amount of the visible operand. Furthermore, by means of the multi-layer calculation of CRBM, it can do a multi-layer calculation towards the complicated sensing data to further obtain a precise classification, such that the percentage of recognizing the target gases becomes more accurate.

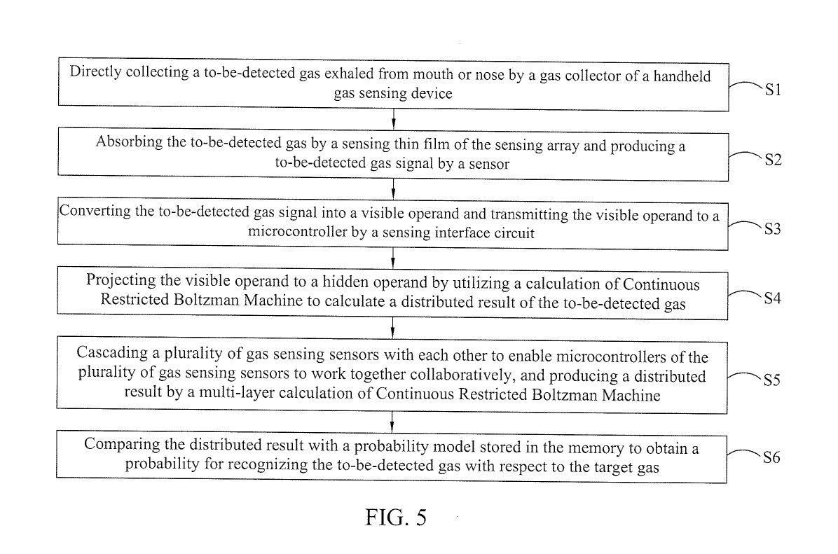

Please refer to FIG. 5, which is a flow chart of the gas sensing method of the present disclosure. As shown in FIG. 5, the gas sensing method includes following steps S1 to S6 and the details are as follows.

Step S1: Directly collecting a to-be-detected gas exhaled from mouth or nose by a gas collector of a handheld gas sensing device. The collected to-be-detected gas is transported to a plurality of gas sensing chips through a transfer pipeline. Each of the plurality of gas sensing chips includes a sensing array, and the plurality of sensing chips corresponds to different target gas, so as to increase the types of sensing gases.

Step S2: Absorbing the to-be-detected gas by a sensing thin film of the sensing array and producing a to-be-detected gas signal by a sensor. The sensing thin film includes a plurality of nanoporous carbon materials and a polymer grows in pores of the nanoporous carbon materials to absorb the to-be-detected gas. The sensor includes a conductive polymer gas sensor and a surface acoustic wave sensor.

Step S3: Converting the to-be-detected gas signal into a visible operand and transmitting the visible operand to a microcontroller by a sensing interface circuit.

Step S4: Projecting the visible operand to a hidden operand by utilizing a calculation of Continuous Restricted Boltzman Machine to calculate a distributed result of the to-be-detected gas.

Step S5: Cascading the plurality of gas sensing sensors with each other to enable microcontrollers of the plurality of gas sensing sensors to work together collaboratively and producing a distributed result by a multi-layer calculation of Continuous Restricted Boltzman Machine. The collaborative working method is producing the hidden operand of one of the gas sensing chips through projection and calculation to serve as the visible operand of another gas sensing chip so as to be projected again to produce another hidden operand. Thereby, the multi-layer calculation of continuous restricted Boltzman machine is completed.

Step S6: Comparing the distributed result with a probability model stored in the memory to obtain a probability for recognizing the to-be-detected gas with respect to the target gas.

In addition to the aforementioned steps, the gas sensing method further includes a temperature-humidity sensor which is provided to produce a temperature-humidity signal to correct the visible operand of the to-be-detected gas, such that it can avoid the sensor being affected due to the temperature and humidity to cause errors. As to the distributed result calculated by the microcontroller, the distributed result is classified by the classifier through a linear programming model or a support vector model to obtain the optimal classified result which is served as the gas recognition.

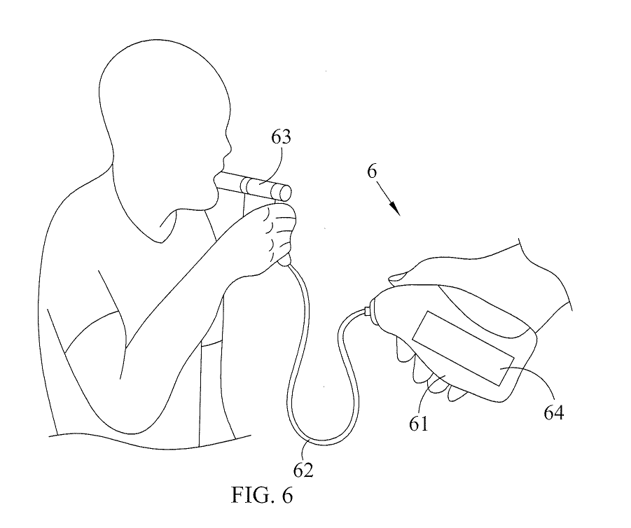

Please refer to FIG. 6 which is a schematic diagram of the handheld gas sensing device of the present disclosure. As shown in FIG. 6, the handheld gas sensing device 6 includes a device main body 61, a transfer pipeline 62 and a blow pipe 63. The device main body 61 includes the plurality of gas sensing chips mentioned in the foregoing embodiment, the blow pipe 63 transfers the gas exhaled from mouth to the gas sensing chips of the device main body 61 through h the transfer pipeline 62. The to-be-detected gas is analyzed by the gas sensing chips to calculate the possible target gases which the to-be-detected gas may contain. Moreover, the sensed result is displayed by a display screen 64 of the device main body 61, and the screen 64 may be a LCD or LED display panel. The display screen 64 is connected to the microcontroller and displays the type and probability for recognizing the to-be-detected gas with respect to the target gas. The display screen 64 may only apply light signals to display the type of the gas. As the handheld gas sensing device 6 is able to be integrated into a handheld device by a manner of system chip or system-in-package, the volume of the device is shortened so as to be carried on the body by the user and increase the practicability. In addition, the chips, which are designed as low-voltage, low power consumption and expandable sensing function, are able to promote the flexibility of the usage of the device.

While the means of specific embodiments in present disclosure has been described by reference drawings, numerous modifications and variations could be made thereto by those skilled in the art without departing from the scope and spirit of the invention set forth in the claims. The modifications and variations should in a range limited by the specification of the present disclosure.

What is claimed is:

1. A handheld gas sensing device, comprising:
a plurality of gas sensing chips, and each of the plurality of gas sensing chips comprising:
a sensing array comprising a sensing thin film and a sensor, the sensing thin film being provided for absorbing gas, such that a to-be-detected gas exhaled from mouth or nose is absorbed and a to-be-detected gas signal is produced by the sensor;
a sensing interface circuit, being connected to the sensing array and converting the to-be-detected gas signal into a visible operand;
a microcontroller, being connected to the sensing interface circuit and projecting the visible operand to a hidden operand by utilizing a calculation of Continuous Restricted Boltzman Machine to calculate a distributed result of the to-be-detected gas, and comparing the distributed result with a probability model of a target gas to obtain a probability for recognizing the to-be-detected gas with respect to the target gas, and
a memory, recording the target gas and the probability model of the target gas, and recording the probability of a comparison result; and
a gas collector, directly collecting the to-be-detected gas exhaled from mouth or nose, and the to-be-detected gas transferred to the sensing array through a transfer pipeline;
wherein, the plurality of gas sensing chips are cascaded to each other, such that the microcontrollers of the plurality of gas sensing chips collaboratively work together, and the hidden operand of one of the gas sensing chips produced through projection and calculation is served as the visible operand of another gas sensing chip so as to be projected again to produce another hidden operand, and the distributed result produced by a multi-layer calculation of Continuous Restricted Boltzman Machine is compared with the probability model to obtain the probability for recognizing the to-be-detected gas with respect to the target gas.

2. The handheld gas sensing device of claim 1, wherein the plurality of gas sensing chips correspond to respective target gases, and the plurality of the gas sensing chips are connected in parallel with each other to simultaneously obtain the probability for recognizing the to-be-detected gas with respect to the respective target gases.

3. The handheld gas sensing device of claim 1, further comprising a temperature-humidity sensor comprising a resistance having a temperature coefficient and a humidity coefficient, and a measured value of the resistance producing a temperature-humidity signal to correct the visible operand of the to-be-detected gas according to the temperature-humidity signal.

4. The handheld gas sensing device of claim 1, wherein the probability model comprises a classifier and the classifier classifies the distributed result and compares the distributed result with the probability model to obtain the probability for recognizing the to-be-detected gas with respect to the target gas.

5. The handheld gas sensing device of claim 1, wherein the classifier classifies the distributed result by a linear programming model or a support vector model.

6. The handheld gas sensing device of claim 1, wherein the sensing thin film comprises a plurality of nanoporous carbon materials and a polymer grows in pores of the nanoporous carbon materials to absorb the to-be-detected gas.

7. The handheld gas sensing device of claim 1, wherein the sensor comprises a conductive polymer gas sensor and a surface acoustic wave sensor.

8. The handheld gas sensing device of claim 1, further comprising a display device to display the probability for recognizing the to-be-detected gas with respect to the target gas.

9. A gas sensing method, comprising following steps:
directly collecting a to-be-detected gas exhaled from mouth or nose by a gas collector of a handheld gas sensing device and transporting the to-be-detected gas to a plurality of gas sensing chips through a transfer pipeline, and each of the plurality of gas sensing chips comprising a sensing array;
absorbing the to-be-detected gas by a sensing thin film of the sensing array and producing a to-be-detected gas signal by a sensor;
converting the to-be-detected gas signal into a visible operand and transmitting the visible operand to a microcontroller by a sensing interface circuit;
projecting the visible operand to a hidden operand by utilizing a calculation of Continuous Restricted Boltzman Machine to calculate a distributed result of the to-be-detected gas;
cascading the plurality of gas sensing sensors with each other to enable microcontrollers of the plurality of gas sensing sensors to work together collaboratively and producing the hidden operand of one of the gas sensing chips through projection and calculation to serve as the visible operand of another gas sensing chip so as to be projected again to produce another hidden operand, and producing the distributed result by a multi-layer calculation of Continuous Restricted Boltzman Machine;
comparing the distributed result with a probability model stored in a memory to obtain a probability for recognizing the to-be-detected gas with respect to a target gas.

10. The gas sensing method of claim 9, wherein the plurality of gas sensing chips correspond to respective target gases, and the plurality of the gas sensing chips are connected in parallel with each other to simultaneously obtain the probability for recognizing the to-be-detected gas with respect to the respective target gases.

11. The gas sensing method of claim 9, further comprising following step:
producing a temperature-humidity signal by a temperature-humidity sensor to correct the visible operand of the to-be-detected gas, and the temperature-humidity sensor comprising a resistance having a temperature coefficient and a humidity coefficient.

12. The gas sensing method of claim 9, wherein the distributed result of the to-be-detected gas is classified by a classifier and the distributed result is compared with the probability model.

13. The gas sensing method of claim 12, wherein the classifier classifies the distributed result by a linear programming model or a support vector model.

14. The gas sensing method of claim 9, wherein the sensing thin film comprises a plurality of nanoporous carbon materials and a polymer grows in pores of the nanoporous carbon materials to absorb the to-be-detected gas.

15. The gas sensing method of claim 9, wherein the sensor comprises a conductive polymer gas sensor and a surface acoustic wave sensor.

16. The gas sensing method of claim 9, wherein the probability for recognizing the to-be-detected gas with respect to the target gas displays by a display device.
